(19) United States
(12) Patent Application Publication
(10) Pub. No.: US 2019/0025328 A1
(43) Pub. Date: Jan. 24, 2019

(54) TROPONIN I AND SOLUBLE UROKINASE RECEPTOR DETECTION FOR DETERMINING THE RISK OF CARDIOVASCULAR DISEASE

(71) Applicants: ABBOTT LABORATORIES, Abbott Park, IL (US); EMORY UNIVERSITY, Atlanta, GA (US); CARDIORISK LLC, San Diego, CA (US)

(72) Inventors: Agim Beshiri, Abbott Park, IL (US); Arshed Ali Quyyumi, Atlanta, GA (US); Sergey Sikora, San Diego, CA (US); Stephen Epstein, Hyattsville, MD (US)

(73) Assignees: ABBOTT LABORATORIES, Abbott Park, IL (US); EMORY UNIVERSITY, Atlanta, GA (US); CARDIORISK LLC, San Diego, CA (US)

(21) Appl. No.: 15/771,210

(22) PCT Filed: Oct. 27, 2016
(86) PCT No.: PCT/US2016/059198

§ 371 Date: Apr. 26, 2018

Related U.S. Application Data
(60) Provisional application No. 62/246,999, filed on Oct. 27, 2015.

(51) Int. Cl. G01N 33/68; G16H 50/30; G16H 50/20

(52) U.S. Cl. CPC G01N 33/6893; G16H 50/30; G16H 50/20; G01N 2333/4703; G01N 2800/54; G01N 2800/60; G01N 2333/70596; G01N 2333/75; G01N 2800/32; G01N 2800/52; G01N 2800/56; G01N 2333/4712

(57) ABSTRACT

Disclosed are systems and methods for detecting the sample concentration of cardiac troponin I (cTnI) and the sample concentration of soluble urokinase receptor (suPAR) to determine if a subject has or is at risk for developing cardiovascular disease or a complication of previously diagnosed cardiovascular disease.

### CROSS-REFERENCE TO RELATED APPLICATIONS

This application claims the benefit of and priority to U.S. Patent Application No. 62/246,999 filed on Oct. 27, 2015, the disclosure of which is expressly incorporated herein by reference in its entirety.

### FIELD

The present disclosure provides systems and methods for detecting the sample concentration of cardiac troponin I (cTnI) and the sample concentration of soluble urokinase receptor (suPAR) to determine if a subject has or is at risk for developing cardiovascular disease or a complication of previously diagnosed cardiovascular disease.

### BACKGROUND

Cardiovascular disease (CVD) accounts for one in every two deaths in the United States and is the number one killer disease in the United States and most European countries. Thus, prevention of cardiovascular disease is an area of major public health importance. A low-fat diet and exercise are recommended to prevent CVD. In addition, a number of therapeutic agents may be prescribed by medical professionals to those individuals who are known to be at risk having CVD. More aggressive therapy, such as administration of multiple medications or surgical intervention may be used in those individuals who are at high risk of having CVD. Since CVD therapies may have adverse side effects, it is desirable to have methods for identifying those individuals who are at risk, particularly those individuals who are at high risk of experiencing an adverse cardiovascular event near term.

### SUMMARY

The present disclosure provides systems and methods for detecting the sample concentration of cardiac troponin I (cTnI) and/or the sample concentration of soluble urokinase receptor (suPAR) to determine if a subject has or is at risk for developing cardiovascular disease or a complication of previously diagnosed cardiovascular disease.

In some embodiments, provided herein are methods comprising: a) testing a biological sample from a subject with: i) a first assay to determine the sample concentration of cardiac troponin I (cTnI), and ii) a second assay to determine the sample concentration of soluble urokinase receptor (suPAR). In some embodiments, provided herein are methods comprising: a) testing a biological sample from a subject with: i) a first assay to determine the sample concentration of cardiac troponin I (cTnI), and ii) a second assay to determine the sample concentration of soluble urokinase receptor (suPAR); and b) comparing the sample concentration of cTnI to a cTnI control concentration, and comparing the sample concentration of suPAR to a suPAR control concentration; wherein a subject whose sample concentrations for both cTnI and suPAR in the sample are elevated as compared to the control concentrations has or is at risk for developing cardiovascular disease or a complication of cardiovascular disease. In some examples, provided herein are methods for diagnosing a subject with or being at risk for developing cardiovascular disease or a complication of cardiovascular disease when concentrations for both cTnI and suPAR in said sample are elevated above a threshold.

In some examples, provided herein are methods comprising: a) testing a biological sample from a subject with: i) a first assay to determine the sample concentration of soluble urokinase receptor (suPAR). In some embodiments, provided herein are methods comprising: a) testing a biological sample from a subject with: i) a first assay to determine the sample concentration of soluble urokinase receptor (suPAR); and b) comparing the sample concentration of suPAR to a suPAR control concentration; wherein a subject whose sample concentration for suPAR in the sample is elevated as compared to the control concentration is at risk for heart failure. In some examples, provided herein are methods for diagnosing a subject with being at risk for heart failure when the concentration for suPAR in said sample is elevated above a threshold.

In certain embodiments, the subject has previously been diagnosed with cardiovascular disease, and wherein the subject has an elevation in one or both the cTnI and suPAR sample concentrations and is at risk for a complication of cardiovascular disease. In other embodiments, the methods further comprise: c) identifying the subject as having an elevation in both cTnI and suPAR sample concentrations, and d) performing at least one of the following: i) treating the subject with a cardiovascular disease (CVD) therapeutic; ii) prescribing the subject a CVD therapeutic; iii) preparing and/or transmitting a report that indicates the subject is at risk for developing cardiovascular disease or at risk for developing a complication of existing cardiovascular disease; iv) diagnosing the subject as at risk for CVD; v) directing the subject to be admitted to a hospital for CVD risk; vi) testing a sample from the subject with one or more CVD risk assays different from the first and second assays; and/or vii) performing a stress test on the subject.

In certain embodiments, provided herein are methods comprising: a) testing a biological sample from a subject with: i) a first assay to determine the sample concentration of cardiac troponin I (cTnI), and ii) a second assay to determine the sample concentration of soluble urokinase receptor (suPAR); and b) comparing said sample concentration of cTnI to a first threshold value of 4.7 pg/ml (or about 4.7 pg/ml), and comparing said sample concentration of suPAR to second threshold value of 3.5 ng/ml (or about 3.5 ng/ml); wherein a subject whose sample concentrations for cTnI is greater than or equal to 4.7 pg/ml and whole sample concentration of suPAR is greater than or equal to 3.5 ng/ml has or is at risk for developing cardiovascular disease or a complication of cardiovascular disease. In particular embodiments, the methods further comprise: c) identifying said subject as having an elevation in both cTnI and suPAR above said threshold values, and d) performing at least one of the following: i) treating said subject with a cardiovascular disease (CVD) therapeutic; ii) prescribing said subject a CVD therapeutic; iii) preparing and/or transmitting a report that indicates said subject is at risk for developing cardiovascular disease or at risk for developing a complication of existing cardiovascular disease; iv) diagnosing said subject as at risk for CVD; v) directing said subject to be admitted to a hospital for CVD risk; vi) testing a sample from said subject with one or more CVD risk assays different from said first and second assays; vii) performing a stress test on said subject.

In some embodiments, the sample is tested with a third assay to detect the level of C-reactive protein (hs-CRP). In other embodiments, the sample is tested with a third assay to detect fibrin degradation products (FDPs). In certain embodiments, the sample is texted with a third assay to detect heat-shock protein-70 (HSP70). In other embodiments, the sample is further tested for hs-CRP levels, FDP levels, and HSP70 levels.

In particular embodiments, the CVD therapeutic is selected from the group consisting of: an antibiotic, a probiotic, an alpha-adrenergic blocking drug, an angiotensin-converting enzyme inhibitor, an antiarrhythmic drug, an anticoagulant, an antiplatelet drug, a thrombolytic drug, a beta-adrenergic blocking drug, a calcium channel blocker, a brain acting drug, a cholesterol-lowering drug, a digitalis drug, a diuretic, a nitrate, a peripheral adrenergic antagonist, and a vasodilator. In other embodiments, the complication is one or more of the following: non-fatal myocardial infarction, stroke, angina pectoris, transient ischemic attacks, congestive heart failure, aortic aneurysm, aortic dissection, and death. In certain embodiments, the sample comprises whole blood, serum, plasma, urine, cerebrospinal fluid, or bronchioalveolar lavage.

In certain embodiments, the first and/or second assay comprises an immunological assay (e.g., ELISA assay). In some embodiments, the first assay comprises a single-molecule detection assay. In additional embodiments, single-molecule detection assay employs the ERENNA™ system.

In certain embodiments, provided herein are systems comprising: a) components of a first assay, wherein the first assay determines the sample concentration of cardiac troponin I (cTnI), and b) components of a second assay, wherein the second assay determines the sample concentration of soluble urokinase receptor (suPAR). In some embodiments, the systems further comprise a computer system, wherein the computer system comprises: i) a computer processor for receiving, processing, and communicating data, ii) a storage component for storing data which contains a reference database containing a cTnI control concentration value and a suPAR control concentration; and iii) a computer program, embedded within the computer processor, which is configured to process the results of the first and second assays in the context of the reference database to determine, as an outcome, if the subject has or is at risk for developing cardiovascular disease or a complication of cardiovascular disease.

In certain embodiments, the methods further comprise the step of characterizing the subject's risk of experiencing a complication of atherosclerotic cardiovascular disease as higher if levels of cTnI and suPAR are both higher than the control values, and lower if the levels of cTnI and suPAR are lower than the control values. In other embodiments, the complication is one or more of the following: non-fatal myocardial infarction, stroke, transient ischemic attack, angina pectoris, transient ischemic attacks, peripheral artery disease, congestive heart failure, cardiomyopathy (ischemic and non-ischemic), aortic aneurysm, aortic dissection, need for revascularization (coronary artery bypass grafting, coronary angioplasty, coronary stenting) and death. In further embodiments, the risk is a risk of experiencing a complication of atherosclerotic cardiovascular disease over the long term, such as within the ensuing three years or longer time points (e.g., four years, five years, six year, seven years, or longer).

### BRIEF DESCRIPTION OF THE FIGURES

The accompanying figures, which are incorporated in and constitute a part of this specification, illustrate several aspects described below.

FIGS. 1A-1C are bar graphs showing plasma suPAR levels stratified by type of HF (FIGS. 1A and 1B) and NYHA class (FIG. 1C). For panel C, P-value reflects the statistical significance for the ANOVA comparing suPAR levels amongst NYHA class. Error bars represent upper and lower 95% confidence intervals.

FIGS. 2A-2C are Kaplan Meier survival curves for all-cause death (FIG. 2A), cardiovascular death (FIG. 2B), and hospitalization for heart failure (HF) (FIG. 2C), stratified by suPAR quartiles in patients with HF.

FIG. 3 is a forest plot depicting hazard ratios for all-cause death and cardiovascular death (P-value for interaction 0.029 and 0.039 respectively) in patients with ischemic and non-ischemic cardiomyopathy.

FIG. 4 is a Kaplan Meier curve showing incident heart failure stratified by suPAR quartiles in patients without heart failure at enrollment (n=3168).

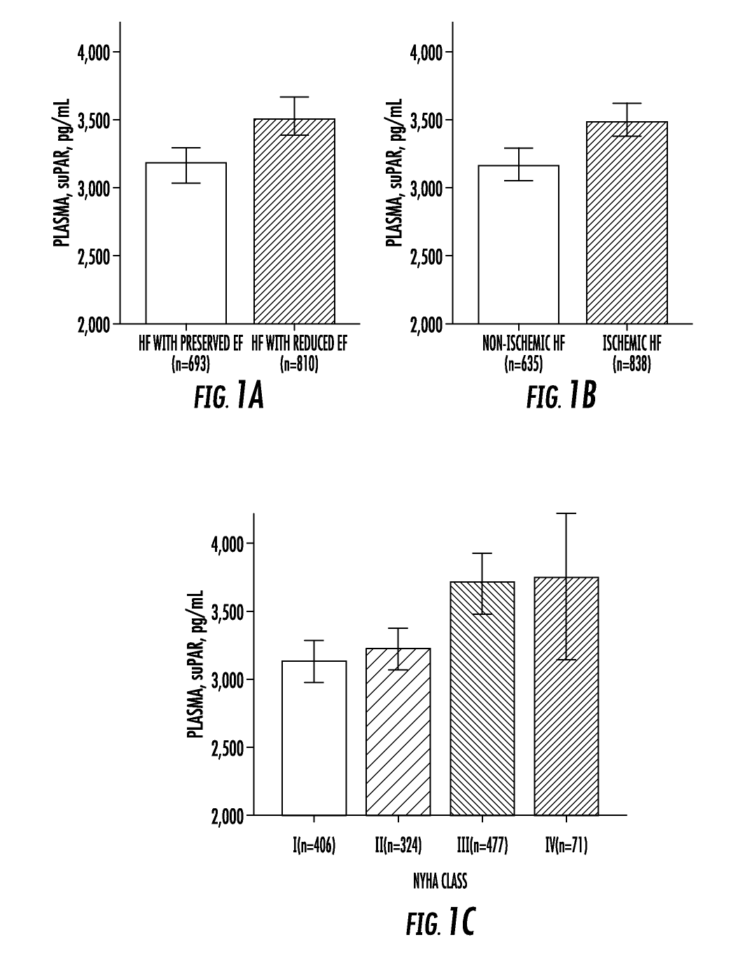

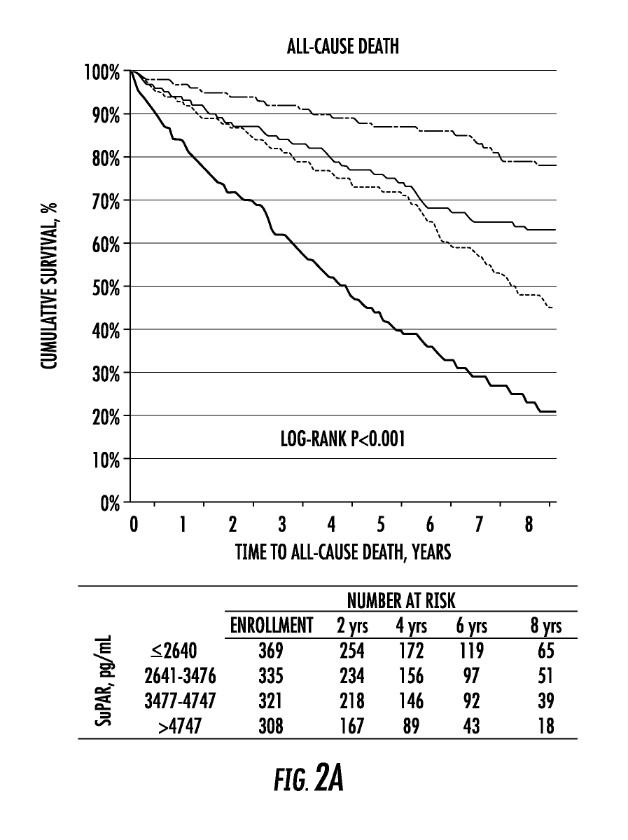

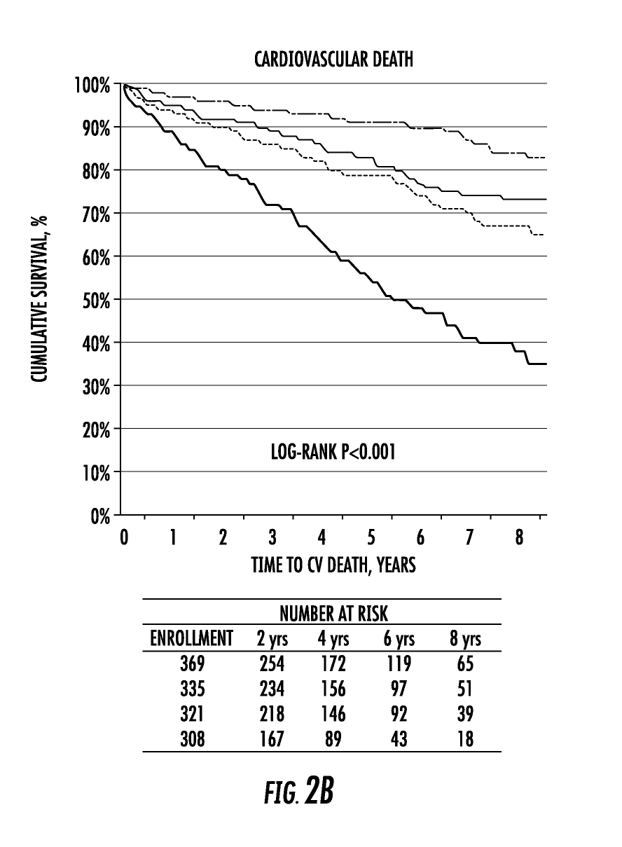

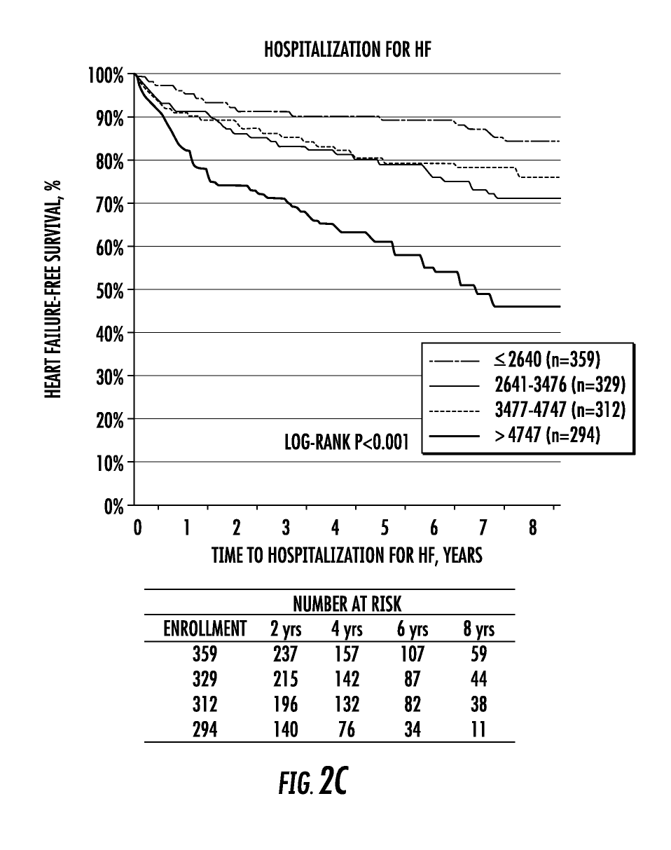

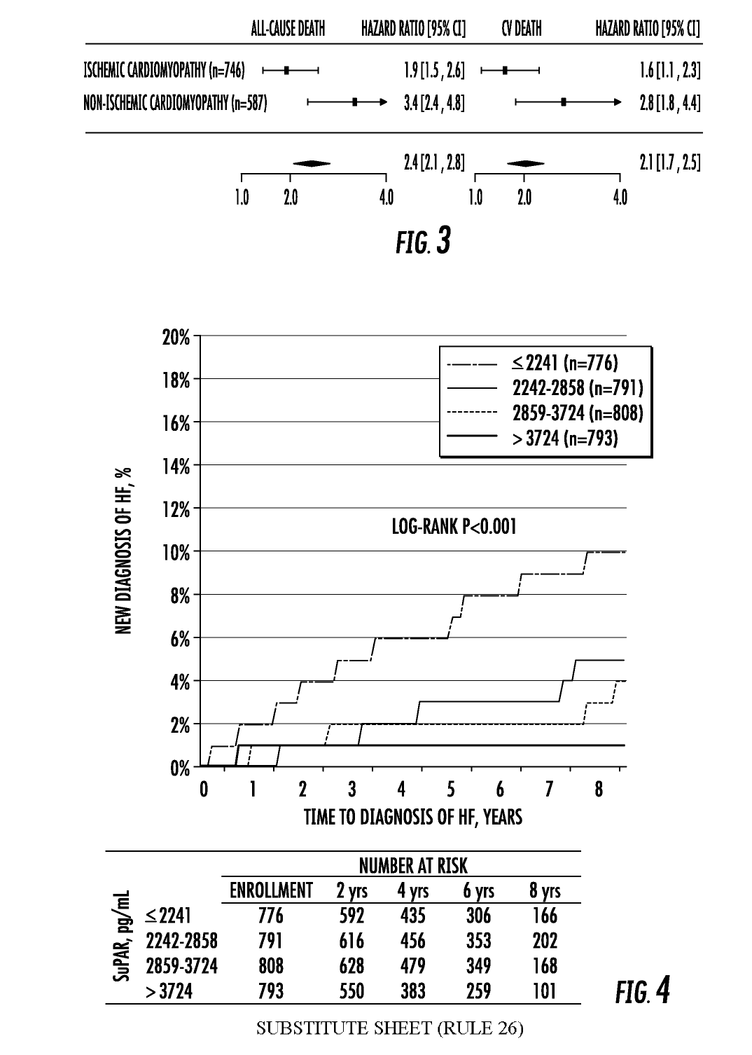

### DETAILED DESCRIPTION

### General Definitions

In this specification and in the claims that follow, reference will be made to a number of terms, which shall be defined to have the following meanings:

As used herein, the terms “cardiovascular disease” (CVD) or “cardiovascular disorder” are terms used to classify numerous conditions affecting the heart, heart valves, and vasculature (e.g., veins and arteries) of the body and encompasses diseases and conditions including, but not limited to arteriosclerosis, atherosclerosis, myocardial infarction, acute coronary syndrome, angina, congestive heart failure, aortic aneurysm, aortic dissection, iliac or femoral aneurysm, pulmonary embolism, primary hypertension, atrial fibrillation, stroke, transient ischemic attack, systolic dysfunction, diastolic dysfunction, myocarditis, atrial tachycardia, ventricular fibrillation, endocarditis, arteriopathy, vasculitis, atherosclerotic plaque, vulnerable plaque, acute coronary syndrome, acute ischemic attack, sudden cardiac death, peripheral vascular disease, coronary artery disease (CAD), peripheral artery disease (PAD), and cerebrovascular disease.

As used herein, the term “atherosclerotic cardiovascular disease” or “disorder” refers to a subset of cardiovascular disease that include atherosclerosis as a component or precursor to the particular type of cardiovascular disease and includes, without limitation, CAD, PAD, cerebrovascular disease. Atherosclerosis is a chronic inflammatory response that occurs in the walls of arterial blood vessels. It involves the formation of atheromatous plaques that can lead to narrowing (“stenosis”) of the artery, and can eventually lead to partial or complete closure of the arterial opening and/or plaque ruptures. Thus atherosclerotic diseases or disorders include the consequences of atheromatous plaque formation and rupture including, without limitation, stenosis or narrowing of arteries, heart failure, aneurysm formation including aortic aneurysm, aortic dissection, and ischemic events such as myocardial infarction and stroke.

A cardiovascular event, as used herein, refers to the manifestation of an adverse condition in a subject brought on by cardiovascular disease, such as sudden cardiac death or acute coronary syndromes including, but not limited to, myocardial infarction, unstable angina, aneurysm, or stroke. The term “cardiovascular event” can be used interchangeably herein with the term cardiovascular complication. While a cardiovascular event can be an acute condition, it can also represent the worsening of a previously detected condition to a point where it represents a significant threat to the health of the subject, such as the enlargement of a previously known aneurysm or the increase of hypertension to life threatening levels.

As used herein, the term “diagnosis” can encompass determining the nature of disease in a subject, as well as determining the severity and probable outcome of disease or episode of disease and/or prospect of recovery (prognosis). “Diagnosis” can also encompass diagnosis in the context of rational therapy, in which the diagnosis guides therapy, including initial selection of therapy, modification of therapy (e.g., adjustment of dose and/or dosage regimen or lifestyle change recommendations), and the like.

The terms “individual,” “host,” “subject,” and “patient” are used interchangeably herein, and generally refer to a mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos. In some embodiments, the subject is specifically a human subject.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, “treatment” refers to obtaining beneficial or desired clinical results. Beneficial or desired clinical results include, but are not limited to, any one or more of: alleviation of one or more symptoms (such as infection), diminishment of extent of infection, stabilized (i.e., not worsening) state of infection, preventing or delaying spread of the infection, preventing or delaying occurrence or recurrence of infection, and delay or slowing of infection progression.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a sample” includes a plurality of such samples and reference to a specific protein includes reference to one or more specific proteins and equivalents thereof known to those skilled in the art, and so forth.

Throughout the specification and claims the word “comprise” and other forms of the word, such as “comprising” and “comprises,” means including but not limited to, and is not intended to exclude, for example, other additives, components, integers, or steps.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values; however, inherently contain certain errors necessarily resulting from error found in their respective measurements.

Reference will now be made in detail to specific aspects of the disclosed materials, compounds, compositions, articles, and methods, examples of which are illustrated in the accompanying Examples and Figures.

### Systems and Methods

The present disclosure provides systems and methods for detecting the sample concentration of cardiac troponin I (cTnI) and the sample concentration of soluble urokinase receptor (suPAR) to determine if a subject has or is at risk for developing cardiovascular disease or a complication of previously diagnosed cardiovascular disease.

The methods of the present invention, in some embodiments, is directed to detection, monitoring, or diagnosis of subjects with regard to specific cardiovascular diseases or cardiovascular events. For example, the methods of the invention can be directed to identifying subjects at risk of developing heart failure or aortic disorders such as aortic aneurysm or aortic dissection.

Heart failure is a form of cardiovascular disease is a condition in which a problem with the structure or function of the heart impairs its ability to supply sufficient blood flow to meet the body's needs, characterized by compromised ventricular systolic or diastolic functions, or both. Heart failure may be manifested by symptoms of poor tissue perfusion alone (e.g., fatigue, poor exercise tolerance, and/or confusion) or by both symptoms of poor tissue perfusion and congestion of vascular beds (e.g., dyspnea, decreased renal function, cardiorenal syndrome, pleural effusion, pulmonary edema, distended neck veins, congested liver, and/or peripheral edema). Congestive heart failure represents a form of heart failure where cardiac output is low, in contrast with high output cardiac failure, in which the body's requirements for oxygen and nutrients are increased, and demand outstrips what the heart can provide.

Heart failure can occur as a result of one or more causes. A major cause is secondary atherosclerotic disease, where one or more ischemic events such as a heart attack result in ischemic injury to the heart and decreased function. This type of heart failure is referred to as ischemic heart failure, because the cause of the cardiac dysfunction was secondary to the ischemic injury. Ischemic heart failure, also sometimes called ischemic cardiomyopathy, can also result from other cardiovascular conditions leading to ischemic injury, such as atherosclerosis that limits blood flow.

Heart failure can also occur as a result of causes other than ischemia, and such forms of heart failure are referred to as non-ischemic heart failure. Examples of non-ischemic heart failure include myocarditis resulting from viral infection, amyloidosis of cardiac tissue, arrhythmia, manifestation of genetic defects, injury from abuse of alcohol, drugs, or cigarettes, other sources of injury to cardiac tissue such as infection by bacteria or parasites, or vitamin deficiency.

Aortic dissection is a tear in the wall of the aorta that causes blood to flow between the layers of the wall of the aorta and force the layers apart. In an aortic dissection, blood penetrates the intima, which is the innermost layer of the aortic artery, and enters the media layer. The high pressure rips the tissue of the media apart along the laminated plane splitting the inner ⅔ and the outer ⅓ of the media apart. This can propagate along the length of the a01ta for a variable distance forward or backwards. Dissections that propagate towards the iliac bifurcation (with the flow of blood) are called anterograde dissections and those that propagate towards the aortic root (opposite of the flow of blood) are called retrograde dissections. The initial tear is usually within 100 mm of the aortic valve so a retrograde dissection can easily compromise the pericardium leading to a hemocardium. Aortic dissection is a medical emergency and can quickly lead to death, even with optimal treatment.

Symptoms of aortic dissection are known to those skilled in the art, and include severe pain that had a sudden onset that may be described as tearing in nature, or stabbing or sharp in character. Some individuals will report that the pain migrates as the dissection extends down the aorta. While the pain may be confused with the pain of a myocardial infarction, aortic dissection is usually not associated with the other signs that suggest myocardial infarction, including heart failure, and ECG changes. Individuals experiencing an aortic dissection usually do not present with diaphoresis (profuse sweating). Individuals with chronic dissection may not indicate the presence of pain. Aortic insufficiency is also typically seen. Other less common symptoms that may be seen in the setting of aortic dissection include congestive heart failure (7%), syncope (9%), cerebrovascular accident (3-6%), ischemic peripheral neuropathy, paraplegia, cardiac arrest, and sudden death. Preferably, this diagnosis is made by visualization of the intimal flap on a diagnostic imaging test such as a CT scan of the chest with iodinated contrast material and a trans-esophageal echocardiogram.

An aortic aneurysm, on the other hand, is a cardiovascular disorder characterized by a swelling of the aorta, which is usually caused by an underlying weakness in the wall of the aorta at that location. Aortic aneurysms are classified by where they occur on the aorta. Abdominal aortic aneurysms, hereafter referred to as AAAs, are the most common type of aortic aneurysm, and are generally asymptomatic before rupture. AAAs are attributed primarily to atherosclerosis, though other factors are involved in their formation. An AAA may remain asymptomatic indefinitely. There is a large risk of rupture once the size has reached 5 cm, though some AAAs may swell to over 15 cm in diameter before rupturing. Only 10-25% of patients survive rupture due to large pre- and post-operative mortality.

Symptoms of an aortic aneurysm may include: anxiety or feeling of stress; nausea and vomiting; clammy skin; rapid heart rate. However, an intact aortic aneurysm may not produce symptoms. As they enlarge, symptoms such as abdominal pain and back pain can develop. Compression of nerve roots may cause leg pain or numbness. Untreated, aneurysms tend to become progressively larger, although the rate of enlargement is unpredictable for a given individual. In some cases, clotted blood which lines most aortic aneurysms can break off and result in an embolus. Preferably, medical imaging is used to confirm the diagnosis of an aortic aneurysm.

In one embodiment, the method is used to assess the test subject's risk of having cardiovascular disease, and in particular atherosclerotic cardiovascular disease, by assessing both cardiac troponin I (cTnI) and suPAR. One form of cardiovascular disease is coronary artery disease. Medical procedures for determining whether a human subject has coronary artery disease or is at risk for experiencing a complication of coronary artery disease include, but are not limited to, coronary angiography, coronary intravascular ultrasound (IVUS), stress testing (with and without imaging), assessment of carotid intimal medial thickening, carotid ultrasound studies with or without implementation of techniques of virtual histology, coronary artery electron beam computer tomography (EBTC), cardiac computerized tomography (CT) scan, CT angiography, cardiac magnetic resonance imaging (MRI), and magnetic resonance angiography (MRA). Because cardiovascular disease is typically not limited to one region of a subject's vasculature, a subject who is diagnosed as having or being at risk of having coronary artery disease is also considered at risk of developing or having other forms of CVD such as cerebrovascular disease, aortic-iliac disease, and peripheral artery disease. Subjects who are at risk of having cardiovascular disease are at risk of having an abnormal stress test or abnormal cardiac catheterization. Subjects who are at risk of having CVD are also at risk of exhibiting increased carotid intimal medial thickness and coronary calcification, characteristics that can be assessed using non-invasive imaging techniques. Subjects who are at risk of having CVD are also at risk of having an increased atherosclerotic plaque burden, a characteristic that can be examined using intravascular ultrasound.

### Biological Samples

Biological samples include, but are not necessarily limited to bodily fluids such as blood-related samples (e.g., whole blood, serum, plasma, and other blood-derived samples), urine, cerebral spinal fluid, bronchioalveolar lavage, and the like. Another example of a biological sample is a tissue sample. A biological sample may be fresh or stored (e.g. blood or blood fraction stored in a blood bank). The biological sample may be a bodily fluid expressly obtained for the assays of this invention or a bodily fluid obtained for another purpose which can be sub-sampled for the assays of this invention. In one embodiment, the biological sample is whole blood. Whole blood may be obtained from the subject using standard clinical procedures. In another embodiment, the biological sample is plasma. Plasma may be obtained from whole blood samples by centrifugation of anti-coagulated blood. Such process provides a buffy coat of white cell components and a supernatant of the plasma. In another embodiment, the biological sample is serum. Serum may be obtained by centrifugation of whole blood samples that have been collected in tubes that are free of anti-coagulant. The blood is permitted to clot prior to centrifugation. The yellowish-reddish fluid that is obtained by centrifugation is the serum. In another embodiment, the sample is urine. The sample may be pretreated as necessary by dilution in an appropriate buffer solution, heparinized, concentrated if desired, or fractionated by any number of methods including but not limited to ultracentrifugation, fractionation by fast performance liquid chromatography (FPLC), or precipitation of apolipoprotein B containing proteins with dextran sulfate or other methods. Any of a number of standard aqueous buffer solutions at physiological pH, such as phosphate, Tris, or the like, can be used.

### Subjects

The subject is any human or other animal to be tested for characterizing its risk of CVD (e.g. congestive heart failure, aortic aneurysm or aortic dissection). In certain embodiments, the subject does not otherwise have an elevated risk of an adverse cardiovascular event, or has previously been diagnosed with CVD. Subjects having an elevated risk of experiencing a cardiovascular event include those with a family history of cardiovascular disease, elevated lipids, smokers, prior acute cardiovascular event, etc. (See, e.g., Harrison's Principles of Experimental Medicine, 15th Edition, McGraw-Hill, Inc., N.Y.—hereinafter “Harrison's”).

In certain embodiments the subject is apparently healthy. “Apparently healthy”, as used herein, describes a subject who does not have any signs or symptoms of CVD, or has not previously been diagnosed as having any signs or symptoms indicating the presence of atherosclerosis, such as angina pectoris, history of a cardiovascular event such as a myocardial infarction or stroke, or evidence of atherosclerosis by diagnostic imaging methods including, but not limited to coronary angiography. Apparently healthy subjects also do not have any signs or symptoms of having heart failure or an aortic disorder.

In other embodiments, the subject already exhibits symptoms of cardiovascular disease. For example, the subject may exhibit symptoms of heart failure or an aortic disorder such as aortic dissection or aortic aneurysm. For subjects already experiencing cardiovascular disease, the levels of cTnI and suPAR, combined, can be used to predict the likelihood of further cardiovascular events or the outcome of ongoing cardiovascular disease.

In certain embodiments, the subject is a nonsmoker. “Nonsmoker” describes an individual who, at the time of the evaluation, is not a smoker. This includes individuals who have never smoked as well as individuals who have smoked but have not used tobacco products within the past year. In certain embodiments, the subject is a smoker.

In some embodiments, the subject is a nonhyperlipidemic subject. “Nonhyperlipidemic” describes a subject that is a nonhypercholesterolemic and/or a nonhypertriglyceridemic subject. A “nonhypercholesterolemic” subject is one that does not fit the current criteria established for a hypercholesterolemic subject. A nonhypertriglyceridemic subject is one that does not fit the current criteria established for a hypertriglyceridemic subject (See, e.g., Harrison's Principles of Experimental Medicine, 15th Edition, McGraw-Hill, Inc., N.Y.—hereinafter “Harrison's”). Hypercholesterolemic subjects and hypertriglyceridemic subjects are associated with increased incidence of premature coronary heart disease. A hypercholesterolemic subject has an LDL level of >160 mg/dL, or >130 mg/dL and at least two risk factors selected from the group consisting of male gender, family history of premature coronary heart disease, cigarette smoking (more than 10 per day), hypertension, low HDL (<35 mg/dL), diabetes mellitus, hyperinsulinemia, abdominal obesity, high lipoprotein (a), and personal history of cerebrovascular disease or occlusive peripheral vascular disease. A hypertriglyceridemic subject has a triglyceride (TG) level of >250 mg/dL. Thus, a nonhyperlipidemic subject is defined as one whose cholesterol and triglyceride levels are below the limits set as described above for both the hypercholesterolemic and hypertriglyceridemic subjects.

### Exemplary Assays

The present disclosure is not limited by the type of assay used to detect cTnI and suPAR, as well as HSP70, hs-CRP, and fibrin degradation products (FDPs). In certain embodiments, the methods for detecting troponin I are as described in U.S. Pat. Pub. 2012/0076803 and U.S. Pat. No. 8,535,895, both of which are herein incorporated by reference, particularly for assay design. In certain embodiments, assays for detecting suPAR are described in EP2115478 and U.S. Pat. Pub. US2010/098705, both of which are herein incorporated by reference, particularly for assay design. In some embodiments, methods for detecting hs-CRP, HSP70, and FDPs are as described in U.S. Pat. Pub. 2014/0350129, herein incorporated by reference, particularly for assay design.

In certain embodiments, an immunoassay is employed for detecting cTnI, suPAR, hs-CRP, HSP70, and/or FDps. In some examples, an immunoassay is employed for detecting cTnI only. In some examples, an immunoassay is employed for detecting suPAR only. In some examples, an immunoassay is employed for detecting cTnI and suPAR. Any suitable assay known in the art can be used. Examples of such assays include, but are not limited to, immunoassay, such as sandwich immunoassay (e.g., monoclonal-polyclonal sandwich immunoassays, including radioisotope detection (radioimmunoassay (RIA)) and enzyme detection (enzyme immunoassay (EIA) or enzyme-linked immunosorbent assay (ELISA) (e.g., Quantikine ELISA assays, R&D Systems, Minneapolis, Minn.)), competitive inhibition immunoassay (e.g., forward and reverse), fluorescence polarization immunoassay (FPIA), enzyme multiplied immunoassay technique (EMIT), bioluminescence resonance energy transfer (BRET), and homogeneous chemiluminescent assay, etc.

If the method involves the determination of the level of more than one of cTnI, suPAR, hs-CRP, HSP70, and/or FDps, the method may be performed on a single sample in parallel (e.g. by multiplex analysis) or on a single sample in sequence (e.g. where the single sample is assayed multiple times, once for each marker that is to be detected or the level thereof determined). Alternatively, the analysis may be performed on multiple (e.g. 2, 3, 4, 5, 6, 7, 8 or at least 2, 3, 4, 5, 6, 7, or 8) samples obtained from the same patient.

### Kits

Also disclosed herein are kits that include a system comprising components of a first assay, wherein the first assay determines the sample concentration of cardiac troponin I (cTnI), and components of a second assay, wherein said second assay determines the sample concentration of soluble urokinase receptor (suPAR). In some embodiments, the kit can include a cup for receiving an biological sample; at least one assay that can detect the levels of cTnI, suPAR, hs-CRP, HSP70, and/or FDps; and optionally reference levels for cTnI, suPAR, hs-CRP, HSP70, and/or FDps, wherein the reference levels are determined by a multivariate analysis or logistic regression calculation using cTnI, suPAR, hs-CRP, HSP70, and/or FDps levels from populations of healthy individuals and individuals with a CVD, and wherein the reference levels delimit different, statistically significant risks for the CVD. In some cases, the assay can be a lateral flow test (lateral flow assay), thus multiple screenings are feasible.

### Control Values

In certain embodiments, levels of cTnI and/or suPAR in the biological sample obtained from the test subject may compared to a control value. A control value is a concentration of an analyte that represents a known or representative amount of an analyte. For example, the control value can be based upon levels of cTnI and/or suPAR in comparable samples obtained from a reference cohort. In certain embodiments, the reference cohort is the general population. In certain embodiments, the reference cohort is a select population of human subjects. In certain embodiments, the reference cohort is comprised of individuals who have not previously had any signs or symptoms indicating the presence of atherosclerosis, such as angina pectoris, history of a cardiovascular event such as a myocardial infarction or stroke, evidence of atherosclerosis by diagnostic imaging methods including, but not limited to coronary angiography. In certain embodiments, the reference cohort includes individuals, who if examined by a medical professional would be characterized as free of symptoms of disease (e.g., cardiovascular disease). For example, a corresponding body sample that originates from a healthy person. In another example, the reference cohort may be individuals who are nonsmokers (i.e., individuals who do not smoke cigarettes or related items such as cigars). A nonsmoker cohort may have a different normal range of cTnI and suPAR than will a smoking population or the general population. Accordingly, the control values selected may take into account the category into which the test subject falls. Appropriate categories can be selected with no more than routine experimentation by those of ordinary skill in the art.

The control value is preferably measured using the same units used to characterize the level of cTnI and/or suPAR obtained from the test subject. The control value can take a variety of forms. The control value can be a single cut-off value, such as a median or mean. The control value can be established based upon comparative groups such as where the risk in one defined group is double the risk in another defined group. The control values can be divided equally (or unequally) into groups, such as a low risk group, a medium risk group and a high-risk group, or into quadrants, the lowest quadrant being individuals with the lowest risk the highest quadrant being individuals with the highest risk, and the test subject's risk of having CVD can be based upon which group his or her test value falls. Control values of cTnI and suPAR in biological samples obtained, such as mean levels, median levels, or “cut-off” levels, are established by assaying a large sample of individuals in the general population or the select population and using a statistical model such as the predictive value method for selecting a positivity criterion or receiver operator characteristic curve that defines optimum specificity (highest true negative rate) and sensitivity (highest true positive rate) as described in Knapp, R. G., and Miller, M. C. 15 (1992). Clinical Epidemiology and Biostatistics. William and Wilkins, Harual Publishing Co. Malvern, Pa., which is specifically incorporated herein by reference. A “cutoff” value can be determined for each risk predictor that is assayed.

Levels of cTnI and suPAR in a subject's biological sample may be compared to a single control value or to a range of control values. If the levels of cTnI and suPAR in the test subject's biological sample are greater than the control values or exceeds or is in the upper range of control values, the test subject is at greater risk of developing or having CVD or experiencing a cardiovascular event within the ensuing year, two years, and/or three years than individuals with levels comparable to or below the control value or in the lower range of control values. In contrast, if levels of cTnI and suPAR in the test subject's biological sample is below the control value or is in the lower range of control values, the test subject is at a lower risk of developing or having CVD or experiencing a cardiovascular event within the ensuing year, two years, and/or three years than individuals whose levels are comparable to or above the control value or exceeding or in the upper range of control values. The extent of the difference between the test subject's risk predictor levels and control value is also useful for characterizing the extent of the risk and thereby determining which individuals would most greatly benefit from certain aggressive therapies. In those cases, where the control value ranges are divided into a plurality of groups, such as the control value ranges for individuals at high risk, average risk, and low risk, the comparison involves determining into which group the test subject's level of the relevant risk predictor falls.

In some embodiments, the levels of cTnI and suPAR in the biological sample require no comparison between the biological sample and a corresponding control that, for example, originates from a healthy person. For example, the levels of cTnI and suPAR indicative of a poor prognosis in a sample may preclude the need for comparison to a corresponding sample that originates from a healthy person.

In some embodiments, disclosed herein are methods of identifying a subject as having an elevation in both cTnI and suPAR above control (threshold) values. In some embodiments, disclosed herein are methods of identifying a subject as having an elevation in of suPAR above a threshold value. The method can include performing at least one of the following: i) treating said subject with a cardiovascular disease (CVD) therapeutic; ii) prescribing said subject a CVD therapeutic; iii) preparing and/or transmitting a report that indicates said subject is at risk for developing cardiovascular disease or at risk for developing a complication of existing cardiovascular disease; iv) diagnosing said subject as at risk for CVD; v) directing said subject to be admitted to a hospital for CVD risk; vi) testing a sample from said subject with one or more CVD risk assays different from said first and second assays; or vii) performing a stress test on said subject.

### EXAMPLES

The following examples are for purposes of illustration only and are not intended to limit the scope of the claims.

### Example 1: Biomarker Risk Score Incorporating Myocardial Injury, Inflammation, Coagulation and Cellular Stress Improves the Prediction of Cardiovascular Events

Introduction:

A multimarker risk score (MRS) including measures of inflammation (C-reactive protein hs-CRP, and soluble urokinase receptor suPAR), coagulation (fibrin degradation products FDP) and cell stress (heat-shock protein-70 HSP70) pathways was shown to be predictive of incident cardiovascular events. We sought to determine whether the addition of high sensitivity troponin-I (hs-TnI)-a marker of myocardial injury—in the MRS further improved risk-stratification in patients with coronary artery disease (CAD).

Methods:

2867 patients without AMI (mean age 62±12, 64% male, 17% black) who underwent cardiac catheterization were recruited into the Emory Biobank. Plasma hs-TnI, and suPAR, and serum hs-CRP, FDP and HSP-70 were measured at enrollment and subjects followed for a median of 3.2 years. The MRS was created by the sum of elevated biomarkers, using previously determined cut-off values. A Cox proportional hazard regression model was used to analyze the outcome of all-cause death, cardiovascular death, MI and hospitalization for heart failure.

Results:

Elevated hs-CRP (≥3.0 mg/L, HR 1.8 [1.4-2.2]), HSP70 (>0.31 ng/mL, HR 1.4 [1.1,1.7]), FDP (≥1.0 μg/mL, HR 1.6 [1.3,2.0]), suPAR (≥3.5 ng/mL, HR 2.3 [1.8,2.9]) and hsTnI (≥4.7 pg/mL, 2.5 [1.9,3.2]) were independently associated with all-cause death in a model incorporating demographics, traditional risk factors, history of MI, CAD severity, eGFR, use of ACE/ARBs, and statins. A higher MRS was associated with worse survival (HR 8.0 [4.4,14.7] vs. 1.5 [0.9,2.5] for MRS of 5 vs 0, respectively). Similar risk prediction was observed for cardiovascular death, MI and HF hospitalizations. Addition of the MRS to a clinical model incorporating risk factors and the aforementioned variables was associated with a significant improvement in C-statistic from 0.64 for the clinical model to 0.81 for clinical +MRS model for all cause death. Net reclassification was similarly improved. The value of MRS for risk prediction was observed in subjects with and without significant CAD.

Conclusion:

A 5-biomarker MRS incorporating hs-CRP, FDP, HSP70, suPAR and hs-TnI improves risk-stratification in subjects with CAD.

### Example 2: Soluble Urokinase Plasminogen Activator Receptor and Cardiovascular Outcomes in Patients with Heart Failure

Introduction:

soluble urokinase-type plasminogen activator receptor (suPAR) is a new biomarker that reflects immune activation and predicts outcomes including incident heart failure (HF) in the general population. In this example, it was postulated that suPAR levels will be elevated in all forms of HF, and be associated with adverse outcomes including incident HF, independent of myocardial-specific and inflammatory markers such as high-sensitivity troponin I (hs-TnI) and high-sensitivity c-reactive protein (hs-CRP).

Methods:

plasma suPAR in 5001 patients undergoing cardiac catheterization and enrolled in the Emory Cardiovascular Biobank was measured. The patients were followed for a median of 5 years for adverse events including death, myocardial infarction, hospitalization for HF, and incident HF. Survival analyses using Cox regression were performed after adjusting for clinical characteristics including coronary artery disease severity, renal function, medications, hs-TnI, and hs-CRP levels. The C-statistic for models with and without traditional risk factors, suPAR, hs-TnI or hs-CRP were calculated.

Results:

1503 patients (30%) with HF (mean age 64±13, 66% male, 26% black, 31% with reduced ejection fraction, 58% with ischemic cardiomyopathy) were identified. The median suPAR level was higher in patients with HF compared to those without HF (3344 [IQR 2540, 4600] vs. 2862 [IQR 2242, 3711] pg/mL, respectively, P<0.001) and correlated with NYHA class (r=0.15, P<0.001) and estimated pulmonary artery systolic pressure (r=0.26, P<0.001). In patients with HF, SuPAR levels (log 2 increase) were independently associated with all-cause death (HR 2.37, 95% CI [1.92-2.92]), cardiovascular death (HR 2.09, 95% CI [1.60, 2.72]), incident myocardial infarction (HR 2.08 95% CI [1.29, 3.35]), and hospitalization for HF (HR 1.70, 95% CI [1.32, 2.20]). Addition of suPAR to risk factors improved the c-statistic for death (A=0.058, 95% CI [0.027, 0.090]) and hospitalization for HF (A=0.062, [95% CI 0.031, 0.093]) and was additive to hs-TnI or hs-CRP. Lastly, in patients without known HF, suPAR levels (log 2 increase) were associated with a 2.02-fold increased risk of incident HF. Results are shown in Tables 1-4.

Conclusions:

suPAR levels are higher in patients with HF, and are predictive of adverse cardiovascular outcomes including incident HF, independent of, and in addition to hs-TnI or hs-CRP levels. As a marker of immune activation, suPAR likely reflects upstream pathologic processes leading to HF.

TABLE 1
Demographics and Clinical Characteristics of the Emory Biobank Cohort Stratified by Diagnosis of Heart Failure

| | No Known Heart | | Heart Failure | | | Heart Failure Type | | | | |
|---|---|---|---|---|---|---|---|---|---|---|
| Variables | Failure (n = 3498) | | (n = 1503) | | P-value^{b} | EF < 45% (n = 468) | | EF ≥ 45% (n = 1035) | | P-value^{c} |
| Age, years | 63 | (12) | 64 | (13) | 0.001 | 65 | (13) | 64 | (13) | 0.37 |
| Male, n (%) | 2179 | (62%) | 992 | (66%) | 0.013 | 323 | (69%) | 669 | (65%) | 0.05 |
| Black race, n (%) | 695 | (20%) | 386 | (26%) | <0.001 | 135 | (29%) | 251 | (24%) | 0.035 |
| Body mass index, kg/m² | 30 | (6) | 30 | (6) | 0.15 | 29 | (6) | 30 | (7) | 0.06 |
| Smoking history, n (%) | 2260 | (65%) | 993 | (66%) | 0.33 | 309 | (66%) | 684 | (66%) | 0.99 |
| Hypertension, n (%) | 2672 | (77%) | 1234 | (82%) | <0.001 | 377 | (81%) | 857 | (83%) | 0.27 |
| Diabetes mellitus, n (%) | 1110 | (32%) | 639 | (44%) | <0.001 | 197 | (43%) | 442 | (44%) | 0.65 |
| Low-density lipoprotein, mg/dL | 96 | (37) | 91 | (37) | <0.001 | 91 | (39) | 91 | (36) | 0.99 |
| High-density lipoprotein, mg/dL | 43 | (13) | 42 | (14) | 0.029 | 41 | (14) | 43 | (14) | 0.015 |
| Estimated glomerular filtration rate, | 75 | (23) | 65 | (25) | <0.001 | 63 | (25) | 66 | (25) | 0.030 |
| mL/min/1.73 m² | | | | | | | | | | |
| Chronic kidney disease stage III, n (%) | 838 | (24%) | 619 | (41%) | <0.001 | 201 | (43%) | 418 | (40%) | 0.37 |
| Myocardial infarction history, n (%) | 715 | (21%) | 631 | (45%) | <0.001 | 236 | (54%) | 395 | (41%) | <0.001 |
| Obstructive coronary artery disease, | 2392 | (74%) | 1145 | (82%) | <0.001 | 355 | (83%) | 790 | (82%) | 0.99 |
| n (%) | | | | | | | | | | |
| Statin therapy, n (%) | 2436 | (70%) | 1062 | (71%) | 0.48 | 328 | (70%) | 734 | (71%) | 0.76 |
| Antiplatelet therapy, n (%) | 2662 | (76%) | 1201 | (80%) | 0.003 | 386 | (83%) | 815 | (79%) | 0.10 |
| ACEi/ARB therapy, n (%) | 1840 | (53%) | 925 | (62%) | <0.001 | 327 | (70%) | 598 | (58%) | <0.001 |
| Beta-blocker therapy, n (%) | 2147 | (61%) | 1144 | (76%) | <0.001 | 394 | (84%) | 750 | (73%) | <0.001 |
| Spironolactone, n (%) | 82 | (2%) | 132 | (9%) | <0.001 | 65 | (14%) | 67 | (7%) | <0.001 |
| Loop diuretics, n (%) | 553 | (16%) | 644 | (43%) | <0.001 | 263 | (56%) | 381 | (37%) | <0.001 |
| SuPAR, pg/mL | 2862 | [2242, 3711] | 3344 | [2540, 4600] | <0.001 | 3529 | [2756, 4865] | 3291 | [2502, 4453] | 0.001 |
| Hs-TnI, pg/mL | 4.7 | [2.7, 11] | 11.2 | [5.3, 34.9] | <0.001 | 16.4 | [7.7, 48.7] | 9.7 | [4.6, 29.1] | <0.001 |
| Hs-CRP, mg/dL^{a} | 2.7 | [1.2, 6.6] | 4.0 | [1.4, 10.0] | <0.001 | 4.9 | [1.9, 11.0] | 3.7 | [1.3, 9.6] | 0.008 |

Values are mean (SD), or n (%) as noted.
Obstructive coronary artery disease is defined as the presence of at least one ≥50% stenotic epicardial artery on angiogram, Plasma suPAR, high sensitivity troponin (hs-TnI) and c-reactive protein (hs-CRP) are reported as median [25^{th}, 75^{th} percentile].
^{a}Hs-CRP was measured in a subset of 3296 patients (2469 without, and 827 with heart failure).
^{b}P-value is for comparison between patients with and without heart failure.
^{c}P-value is for comparison between patients with EF < 45% and those with EF ≥ 45%.

TABLE 2
SuPAR and Outcomes in Patients with Heart Failure

| | | | | | Myocardial | | | |
|---|---|---|---|---|---|---|---|---|
| | All-cause death | | CV death | | infarction | | Hospitalization for HF | |
| Variables | HR | 95% CI | HR | 95% CI | HR | 95% CI | HR | 95% CI |
| Model 1: Clinical Characteristics | | | | | | | | |
| Age, per 10 years | 1.30 | 1.15, 1.45 | 1.19 | 1.04, 1.37 | 1.00 | 0.79, 1.27 | 1.15 | 1.01, 1.31 |
| Male | 1.14 | 0.88, 1.47 | 1.07 | 0.78, 1.46 | 1.13 | 0.63, 2.01 | 0.81 | 0.60, 1.10 |
| Black race | 1.06 | 0.78, 1.44 | 1.12 | 0.77, 1.62 | 1.02 | 0.53, 1.95 | 1.51 | 1.07, 2.13 |
| Body mass index, per 5 kg/m² | 0.97 | 0.88, 1.08 | 0.99 | 0.88, 1.13 | 1.15 | 0.94, 1.41 | 0.96 | 0.84, 1.08 |
| Smoking history | 1.49 | 1.15, 1.94 | 1.73 | 1.23, 2.42 | 1.27 | 0.71, 2.28 | 1.40 | 1.01, 1.94 |
| Hypertension | 1.17 | 0.86, 1.59 | 1.12 | 0.76, 1.63 | 1.35 | 0.62, 2.93 | 0.67 | 0.47, 0.97 |
| Diabetes mellitus | 1.11 | 0.87, 1.42 | 1.04 | 0.77, 1.42 | 1.77 | 1.03, 3.06 | 1.62 | 1.19, 2.20 |
| Estimated glomerular filtration rate, per 10 mL/min/ | 0.84 | 0.79, 0.89 | 0.81 | 0.75, 0.86 | 0.85 | 0.76, 0.96 | 0.88 | 0.82, 0.93 |
| 1.73 m² | | | | | | | | |
| Myocardial infarction history | 1.01 | 0.79, 1.29 | 1.00 | 0.74, 1.36 | 1.78 | 1.02, 3.10 | 1.12 | 0.82, 1.52 |
| Obstructive coronary artery disease | 1.15 | 0.81, 1.63 | 1.12 | 0.73, 1.73 | 5.09 | 1.18, 21.98 | 0.94 | 0.62, 1.43 |
| Ejection Fraction, per 5% | 0.92 | 0.88, 0.96 | 0.88 | 0.83, 0.92 | 0.99 | 0.90, 1.09 | 0.90 | 0.85, 0.94 |
| Antiplatelet therapy | 0.72 | 0.51, 1.02 | 0.71 | 0.46, 1.11 | 0.56 | 0.25, 1.23 | 0.53 | 0.35, 0.79 |
| Statin therapy | 0.65 | 0.49, 0.87 | 0.77 | 0.53, 1.10 | 1.58 | 0.70, 3.57 | 0.94 | 0.65, 1.36 |
| ACEi/ARB therapy | 0.75 | 0.58, 0.98 | 0.69 | 0.50, 0.94 | 0.77 | 0.43, 1.37 | 0.70 | 0.51, 0.97 |
| Beta-blocker therapy | 0.93 | 0.68, 1.26 | 1.03 | 0.69, 1.52 | 1.13 | 0.54, 2.37 | 0.99 | 0.67, 1.49 |
| Loop diuretics | 1.87 | 1.46, 2.40 | 2.14 | 1.57, 2.92 | 1.72 | 1.01, 2.93 | 2.14 | 1.57, 2.92 |
| Spironolactone | 0.64 | 0.41, 1.00 | 0.66 | 0.38, 1.12 | 0.51 | 0.18, 1.42 | 1.35 | 0.88, 2.09 |
| Model 2-5: Clinical Characteristics + SuPAR | | | | | | | | |
| SuPAR, per 100% increase (log2) | 2.37 | 1.92, 2.92 | 2.09 | 1.60, 2.72 | 2.08 | 1.29, 3.35 | 1.70 | 1.32, 2.20 |
| SuPAR > 3476 pg/mL | 2.04 | 1.56, 2.67 | 1.77 | 1.27, 2.46 | 2.25 | 1.24, 4.11 | 1.30 | 0.94, 1.79 |
| SuPAR Quartiles | | | | | | | | |
| ≤2640 pg/mL, reference | — | — | — | — | — | — | | |
| 2641-3476 pg/mL | 2.11 | 1.40, 3.18 | 2.11 | 1.28, 3.49 | 1.66 | 0.70, 3.95 | 1.86 | 1.18, 2.93 |
| 3477-4747 pg/mL | 2.54 | 1.67, 3.84 | 2.30 | 1.38, 3.83 | 2.78 | 1.20, 6.48 | 1.48 | 0.92, 2.39 |
| >4747 pg/mL | 4.58 | 2.99, 7.01 | 3.79 | 2.23, 6.45 | 3.52 | 1.41, 8.77 | 2.45 | 1.51, 3.96 |
| Model 6: SuPAR and hs-TnI | | | | | | | | |
| SuPAR, per 100% increase (log2) | 2.29 | 1.82, 2.88 | 2.07 | 1.55, 2.76 | 2.31 | 1.36, 3.92 | 1.72 | 1.30, 2.27 |
| Hs-TnI, per 100% increase (log2) | 1.12 | 1.06, 1.19 | 1.13 | 1.05, 1.21 | 1.20 | 1.07, 1.35 | 1.12 | 1.05, 1.20 |
| Model 7: SuPAR and hs-CRP^{a} | | | | | | | | |
| SuPAR, per 100% increase (log2) | 2.53 | 1.92, 3.35 | 2.15 | 1.51, 3.05 | 2.46 | 1.25, 4.81 | 2.16 | 1.51, 3.08 |
| Hs-CRP, per 100% increase (log2) | 1.12 | 1.04, 1.20 | 1.12 | 1.02, 1.22 | 0.97 | 0.81, 1.15 | 1.18 | 1.07, 1.30 |

^{a}Model 7 was limited to the subset of patients (n = 803) with heart failure, available high sensitivity c-reactive protein (hs-CRP) measurements and at least 30 days follow-up.

TABLE 3
Risk Discrimination Metrics for All-cause Death and Hospitalization for HF in Patients with HF

| | All-cause Death | | | Hospitalization for HF | | |
|---|---|---|---|---|---|---|
| Model | C-statistic (95% CI) | ΔC-statistic (95% CI) | | C-statistic (95% CI) | ΔC-statistic (95% CI) | |
| Models 1-4 (n = 1333) | | | | | | |
| Model 1: Risk factors only | 0.663 (0.621, 0.704) | — | | 0.646 (0.599, 0.694) | — | |
| Model 2: RF and suPAR | 0.721 (0.679, 0.763) | 0.058 | (0.027, 0.090) | 0.674 (0.626, 0.722) | 0.028 | (−0.002, 0.057) |
| Model 3: RF and hs-TnI | 0.676 (0.632, 0.721) | 0.014 | (−0.001, 0.029) | 0.668 (0.620, 0.716) | 0.022 | (−0.001, 0.045) |
| Model 4: RF, suPAR and hs-TnI | 0.725 (0.687, 0.762) | 0.062 | (0.031, 0.093) | 0.687 (0.637, 0.736) | 0.040 | (0.008, 0.072) |
| Models 5-8 (n = 803) | | | | | | |
| Model 5: Risk factors only | 0.666 (0.618, 0.713) | — | | 0.658 (0.595, 0.722) | — | |
| Model 6: RF and suPAR | 0.729 (0.690, 0.769) | 0.064 | (0.029, 0.099) | 0.701 (0.644, 0.759) | 0.043 | (0.001, 0.085) |
| Model 7: RF and hs-CRP | 0.682 (0.633, 0.731) | 0.016, | (−0.006, 0.038) | 0.694 (0.645, 0.743) | 0.036 | (−0.002, 0.073) |
| Model 8: RF, suPAR and hs-CRP | 0.728 (0.681, 0.776) | 0.063, | (0.022, 0.103) | 0.719 (0.666, 0.772) | 0.061 | (0.012, 0.110) |

Models 1 and 5 includes age, gender, race, body mass index, smoking history, hypertension, diabetes, estimated glomerular filtration rate at enrollment, history of myocardial infarction and obstructive CAD (RF). Models 2 and 6 include aforementioned risk factors in Model 1 in addition to suPAR levels. Model 3 includes RF and high sensitivity troponin I (hs-TnI). Model 7 includes RF and high sensitivity c-reactive protein (hs-CRP) Lastly Models 4 and 8 includes RF, suPAR and hs-TnI or hs-CRP respectively. The change in C-statistic, reported is relative to the RF only model. CI: confidence interval

TABLE 4
SuPAR and Incident Heart Failure

| | New diagnosis or hospital- | |
|---|---|---|
| | ization for HF | |
| Variables | HR | 95% CI |
| Model 1: Clinical Characteristics | | |
| Age, per 10 years | 1.24 | 0.96, 1.60 |
| Male | 0.63 | 0.38, 1.04 |
| Black race | 1.14 | 0.59, 2.22 |
| Body mass index, per 5 kg/m² | 1.08 | 0.89, 1.32 |
| Smoking history | 1.12 | 0.67, 1.90 |
| Hypertension | 1.01 | 0.56, 1.83 |
| Diabetes mellitus | 1.66 | 0.99, 2.77 |
| Estimated glomerular filtration rate, per | 0.80 | 0.72, 0.90 |
| 10 mL/min/1.73 m² | | |
| Myocardial infarction history | 1.13 | 0.64, 2.01 |
| Obstructive coronary artery disease | 1.13 | 0.60, 2.11 |
| Antiplatelet therapy | 0.92 | 0.79, 1.07 |
| Statin therapy | 1.41 | 0.64, 3.08 |
| ACEi/ARB therapy | 1.60 | 0.78, 3.31 |
| Beta-blocker therapy | 0.83 | 0.49, 1.40 |
| Model 2-5: Clinical Characteristics + SuPAR | | |
| SuPAR, per 100% increase (log2) | 2.02 | 1.34, 3.05 |
| SuPAR > 2858 pg/mL | 1.97 | 1.10, 3.55 |
| SuPAR Quartiles | | |
| ≤2241 pg/mL, reference | | |
| 2242-2858 pg/mL | 1.32 | 0.51, 3.44 |
| 2859-3724 pg/mL | 1.75 | 0.70, 4.35 |
| >3724 pg/mL | 3.25 | 1.31, 8.06 |
| Model 6: SuPAR and hs-TnI | | |
| SuPAR, per 100% increase (log2) | 1.72 | 1.13, 2.61 |
| Hs-TnI, per 100% increase (log2) | 1.17 | 1.05, 1.31 |
| Model 7: SuPAR and hs-CRP | | |
| SuPAR, per 100% increase (log2) | 1.76 | 1.08, 2.85 |
| Hs-CRP, per 100% increase (log2) | 1.08 | 0.93, 1.28 |

^{a} Model 7 was limited to the subset of patients (n = 2382) without heart failure, but with available high sensitivity c-reactive protein (hs-CRP) measurements and at least 30-days follow-up.

1. A method comprising:
a) testing a biological sample from a subject with: i) a first assay to determine the sample concentration of cardiac troponin I (cTnI), and ii) a second assay to determine the sample concentration of soluble urokinase receptor (suPAR); and
b) diagnosing the subject with or being at risk for developing cardiovascular disease or a complication of cardiovascular disease when concentrations for both cTnI and suPAR in said sample are elevated above a threshold.

2. The method of claim 1, further comprising comparing said sample concentration of cTnI to a cTnI control concentration, and comparing said sample concentration of suPAR to a suPAR control concentration, wherein a subject whose sample concentrations for both cTnI and suPAR in said sample are elevated as compared to said control concentrations has or is at risk for developing cardiovascular disease or a complication of cardiovascular disease.

3. The method of claim 1, wherein said subject has previously been diagnosed with cardiovascular disease, and wherein said subject has an elevation in both said cTnI and suPAR sample concentrations and is at risk for a complication of cardiovascular disease.

4. The method of claim 1, further comprising c) identifying said subject as having an elevation in both cTnI and suPAR sample concentrations, and d) performing at least one of the following:
i) treating said subject with a cardiovascular disease (CVD) therapeutic;
ii) prescribing said subject a CVD therapeutic;
iii) preparing and/or transmitting a report that indicates said subject is at risk for developing cardiovascular disease or at risk for developing a complication of existing cardiovascular disease;
iv) diagnosing said subject as at risk for CVD;
v) directing said subject to be admitted to a hospital for CVD risk;
vi) testing a sample from said subject with one or more CVD risk assays different from said first and second assays; or
vii) performing a stress test on said subject.

5. The method of claim 4, wherein said CVD therapeutic is selected from the group consisting of: an antibiotic, a probiotic, an alpha-adrenergic blocking drug, an angiotensin-converting enzyme inhibitor, an antiarrhythmic drug, an anticoagulant, an antiplatelet drug, a thrombolytic drug, a beta-adrenergic blocking drug, a calcium channel blocker, a brain acting drug, a cholesterol-lowering drug, a digitalis drug, a diuretic, a nitrate, a peripheral adrenergic antagonist, and a vasodilator.

6. The method of claim 1, wherein said complication is one or more of the following: non-fatal myocardial infarction, stroke, angina pectoris, transient ischemic attacks, congestive heart failure, aortic aneurysm, aortic dissection, and death.

7. The method of claim 1, wherein said sample comprises whole blood, serum, plasma, urine, cerebrospinal fluid, or bronchioalveolar lavage.

8. The method of claim 1, wherein said first assay comprises an ELISA assay.

9. The method of claim 1, wherein said first assay comprises a single-molecule detection assay.

10. The method of claim 1, wherein said sample is tested with a third assay to detect the level of C-reactive protein (hs-CRP).

11. The method of claim 1, wherein said sample is tested with a third assay to detect fibrin degradation products (FDPs).

12. The method of claim 1, wherein said sample is tested with a third assay to detect heat-shock protein-70 HSP70.

13. A system comprising:
a) components of a first assay, wherein the first assay determines the sample concentration of cardiac troponin I (cTnI), and
b) components of a second assay, wherein said second assay determines the sample concentration of soluble urokinase receptor (suPAR).

14. The system of claim 13, further comprising a computer system, wherein said computer system comprises: i) a computer processor for receiving, processing, and communicating data, ii) a storage component for storing data which contains a reference database containing a cTnI control concentration and a suPAR control concentration; and iii) a computer program, embedded within said computer processor, which is configured to process said results of said first and second assays in the context of said reference database to determine, as an outcome, if said subject has or is at risk for developing cardiovascular disease or a complication of cardiovascular disease.

15. A method comprising:
a) testing a biological sample from a subject with: i) a first assay to determine the sample concentration of cardiac troponin I (cTnI), and ii) a second assay to determine the sample concentration of soluble urokinase receptor (suPAR); and
b) diagnosing the subject with or being at risk for developing cardiovascular disease or a complication of cardiovascular disease when a subject whose sample concentrations for cTnI is greater than or equal to 4.7 pg/ml and whole sample concentration of suPAR is greater than or equal to 3.5 ng/ml.

16. The method of claim 15, further comprising comparing said sample concentration of cTnI to a first threshold value of 4.7 pg/ml, and comparing said sample concentration of suPAR to a second threshold value of 3.5 ng/ml, wherein a subject whose sample concentrations for cTnI is greater than or equal to 4.7 pg/ml and whole sample concentration of suPAR is greater than or equal to 3.5 ng/ml has or is at risk for developing cardiovascular disease or a complication of cardiovascular disease.

17. The method of claim 15, further comprising c) identifying said subject as having an elevation in both cTnI and suPAR above said threshold values, and d) performing at least one of the following:
i) treating said subject with a cardiovascular disease (CVD) therapeutic;
ii) prescribing said subject a CVD therapeutic;
iii) preparing and/or transmitting a report that indicates said subject is at risk for developing cardiovascular disease or at risk for developing a complication of existing cardiovascular disease;
iv) diagnosing said subject as at risk for CVD;
v) directing said subject to be admitted to a hospital for CVD risk;
vi) testing a sample from said subject with one or more CVD risk assays different from said first and second assays;
vii) performing a stress test on said subject.

18. The method of claim 15, wherein said CVD therapeutic is selected from the group consisting of: an antibiotic, a probiotic, an alpha-adrenergic blocking drug, an angiotensin-converting enzyme inhibitor, an antiarrhythmic drug, an anticoagulant, an antiplatelet drug, a thrombolytic drug, a beta-adrenergic blocking drug, a calcium channel blocker, a brain acting drug, a cholesterol-lowering drug, a digitalis drug, a diuretic, a nitrate, a peripheral adrenergic antagonist, and a vasodilator.

19. The method of claim 15, wherein said complication is one or more of the following: non-fatal myocardial infarction, stroke, angina pectoris, transient ischemic attacks, congestive heart failure, aortic aneurysm, aortic dissection, and death.

20. The method of claim 15, wherein said sample comprises whole blood, serum, plasma, urine, cerebrospinal fluid, or bronchioalveolar lavage.

21. The method of claim 15, wherein said first assay comprises an ELISA assay.

22. The method of claim 15, wherein said first assay comprises a single-molecule detection assay.

23. A method comprising:
a) testing a biological sample from a subject with a first assay to determine the sample concentration of soluble urokinase receptor (suPAR); and
b) diagnosing the subject with being at risk for heart failure when a subject whose sample concentration for suPAR in said sample is elevated above a threshold.

24. The method of claim 23, further comprising comparing said sample concentration of suPAR to a suPAR control concentration, wherein a subject whose sample concentration for suPAR in said sample is elevated as compared to said control concentration is at risk for heart failure.

25. The method of claim 23, further comprising c) identifying said subject as having an elevation in suPAR sample concentrations, and d) performing at least one of the following:
i) treating said subject with a cardiovascular disease (CVD) therapeutic;
ii) prescribing said subject a CVD therapeutic;
iii) preparing and/or transmitting a report that indicates said subject is at risk for developing heart failure;
iv) diagnosing said subject as at risk for heart failure;
v) directing said subject to be admitted to a hospital for heart failure risk;
vi) testing a sample from said subject with one or more heart failure risk assays different from said first assay; or
vii) performing a stress test on said subject.

26. The method of claim 23, wherein said sample comprises whole blood, serum, plasma, urine, cerebrospinal fluid, or bronchioalveolar lavage.

27. The method of claim 23, wherein said sample is tested with a second assay to detect the level of cardiac troponin I (cTnI).

28. The method of claim 23, wherein said sample is tested with a second assay to detect the level of C-reactive protein (hs-CRP).

29. A method comprising:
a) obtaining a biological sample from a subject; and
b) testing the biological sample with: i) a first assay to determine the sample concentration of cardiac troponin I (cTnI), and ii) a second assay to determine the sample concentration of soluble urokinase receptor (suPAR).

30. The method of claim 29, further comprising b) comparing said sample concentration of cTnI to a cTnI control concentration, and comparing said sample concentration of suPAR to a suPAR control concentration, wherein a subject whose sample concentrations for both cTnI and suPAR in said sample are elevated as compared to said control concentrations has or is at risk for developing cardiovascular disease or a complication of cardiovascular disease.

31. The method of claim 29, further comprising b) comparing said sample concentration of cTnI to a first threshold value of 4.7 pg/ml, and comparing said sample concentration of suPAR to a second threshold value of 3.5 ng/ml, wherein a subject whose sample concentrations for cTnI is greater than or equal to 4.7 pg/ml and whole sample concentration of suPAR is greater than or equal to 3.5 ng/ml has or is at risk for developing cardiovascular disease or a complication of cardiovascular disease.

32. The method of claim 29, further comprising d) identifying a subject as having an elevation in both cTnI and suPAR sample concentrations.

33. The method of claim 32, further comprising e) performing at least one of the following:
i) treating said subject with a cardiovascular disease (CVD) therapeutic;
ii) prescribing said subject a CVD therapeutic;
iii) preparing and/or transmitting a report that indicates said subject is at risk for developing cardiovascular disease or at risk for developing a complication of existing cardiovascular disease;
iv) diagnosing said subject as at risk for CVD;
v) directing said subject to be admitted to a hospital for CVD risk;
vi) testing a sample from said subject with one or more CVD risk assays different from said first and second assays; or
vii) performing a stress test on said subject.

34. The method of claim 29, comprising diagnosing a subject with or being at risk for developing cardiovascular disease or a complication of cardiovascular disease when concentrations for both cTnI and suPAR in said sample are elevated above a threshold.
